SEQUENCE LISTING (36 sequences)

SEQ ID NO: 1 - DNA, 21 bp, Artificial Sequence
  SRE control elements used in the promoters.
gacacccaaatatggcgacgg

SEQ ID NO: 2 - DNA, 19 bp, Artificial Sequence
  MEF-1 control element used in the promoters.
ccaacacctgctgcctgcc

SEQ ID NO: 3 - DNA, 19 bp, Artificial Sequence
  MEF-2 control element used in the promoters.
cgctctaaaaataactccc

SEQ ID NO: 4 - DNA, 13 bp, Artificial Sequence
  TEF-1 control element used in the promoters.
caccattcctcac

SEQ ID NO: 5 - DNA, 335 bp, Artificial Sequence
  Nucleic acid sequence of an eukaryotic promoter c5-c12.
cggccgtccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctaga

SEQ ID NO: 6 - protein, 40 aa, Artificial Sequence
  This is the artificial sequence for GHRH (1-40) OH.
XXDAIFTNSYRKVLXQLSARKLLQDIXXRQQGEXNXEXGA

SEQ ID NO: 7 - DNA, 3534 bp, Artificial Sequence
  Nucleic acid sequence for the HV-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggcacgtagatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 8 - DNA, 3534 bp, Artificial Sequence
  Nucleic acid sequence for the TI-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatatcgatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 9 - DNA, 3534 bp, Artificial Sequence
  Nucleic acid sequence for the TV-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatgtagatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 10 - DNA, 3534 bp, Artificial Sequence; identical to SEQ ID NO: 8
  Nucleic acid sequence for the 15/27/28 GHRH plasmid.

SEQ ID NO: 11 - DNA, 2710 bp, Artificial Sequence
  Vector with a mouse codon optimized GHRH analog sequence.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctctgggtgctctttgtgatcctcatcctcaccagcggcagccactgcagcctgcctcccagccctcccttcaggatgcagaggcacgtggacgccatcttcaccaccaactacaggaagctgctgagccagctgtacgccaggaaggtgatccaggacatcatgaacaagcagggcgagaggatccaggagcagagggccaggctgagctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 12 - DNA, 2713 bp, Artificial Sequence
  Vector with a rat codon optimized GHRH analog sequence.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggccctgtgggtgttcttcgtgctgctgaccctgaccagcggaagccactgcagcctgcctcccagccctcccttcagggtgcgccggcacgccgacgccatcttcaccagcagctacaggaggatcctgggccagctgtacgctaggaagctcctgcacgagatcatgaacaggcagcagggcgagaggaaccaggagcagaggagcaggttcaactgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 13 - DNA, 2704 bp, Artificial Sequence
  Vector with a bovine codon optimized GHRH analog sequence.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctgtgggtgttcttcctggtgaccctgaccctgagcagcggctcccacggctccctgccctcccagcctctgcgcatccctcgctacgccgacgccatcttcaccaacagctaccgcaaggtgctcggccagctcagcgcccgcaagctcctgcaggacatcatgaaccggcagcagggcgagcgcaaccaggagcagggagcctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 14 - DNA, 2704 bp, Artificial Sequence
  Vector with a ovine codon optimized GHRH analog sequence.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctgtgggtgttcttcctggtgaccctgaccctgagcagcggaagccacggcagcctgcccagccagcccctgaggatccctaggtacgccgacgccatcttcaccaacagctacaggaagatcctgggccagctgagcgctaggaagctcctgcaggacatcatgaacaggcagcagggcgagaggaaccaggagcagggcgcctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 15 - DNA, 2713 bp, Artificial Sequence
  Vector with a chicken codon optimized GHRH analog sequence.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggccctgtgggtgttctttgtgctgctgaccctgacctccggaagccactgcagcctgccacccagcccacccttccgcgtcaggcgccacgccgacggcatcttcagcaaggcctaccgcaagctcctgggccagctgagcgcacgcaactacctgcacagcctgatggccaagcgcgtgggcagcggactgggagacgaggccgagcccctgagctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 16 - DNA, 382 bp, Artificial Sequence
  This is the synthetic promoter c1-26.
ggcggccgagggcggcggggcaggcagcaggtgttggcaccattcctcaccgctctaaaaataactcccgtgaggaatggtgccgtcgccatatttgggtgtcgacacccaaatatggcgacgggtgaggaatggtgggcaggcagcaggtgttgggacacccaaatatggcgacggccaacacctgctgcctgccgggagttatttttagagcggggagttatttttagagcggtgaggaatggtggacacccaaatatggcgacggccggggccgcattcctgggggccgggcggtgctcccgcccgcctcgataaaaggctccggggccggcggcggcccacgagctacccggaggagcgggaggcgccaagctctaga

SEQ ID NO: 17 - DNA, 218 bp, Artificial Sequence
  This is the synthetic promoter sequence for c2-26.
cggccgtcgccatatttgggtgtccgctctaaaaataactcccgacacccaaatatggcgacggggcaggcagcaggtgttgggacacccaaatatggcgacggccggggccgcattcctgggggccgggcggtgctcccgcccgcctcgataaaaggctccggggccggcggcggcccacgagctacccggaggagcgggaggcgccaagctctaga

SEQ ID NO: 18 - DNA, 230 bp, Artificial Sequence
  This is the snythetic sequence for c2-27.
cggccgtcgccatatttgggtgtcggcaggcagcaggtgttggcaccattcctcacccgtcgccatatttgggtgtcggcaggcagcagtgttgggacacccaaatatggcgacggccggggccgcattcctgggggccgggcggtgctcccgcccgcctcgataaaaggctccggggccggcggcggcccacgagctacccggaggagcgggaggcgccaagctctaga

SEQ ID NO: 19 - DNA, 231 bp, Artificial Sequence
  This is the synthetic promoter for c5-5.
cggccgtccgccctcgggacacccaaatatggcgacgggtgaggaatggtgcaccattcctcacgggagttatttttagagcggtgaggaatggtggacacccaaatatggcgacggccggggccgcattcctgggggccgggcggtgctcccgcccgcctcgataaaaggctccggggccggcggcggcccacgagctacccggaggagcgggaggcgccaagctctaga

SEQ ID NO: 20 - DNA, 255 bp, Artificial Sequnence
  This is the synthetic promoter for c6-6.
cggccgtcgccatatttgggtgtcccaacacctgctgcctgccccgtcgccatatttggtgtcggcaggcagcaggtgttggccaacacctgctgcctgccgggagttatttttagagcggacacccaaatatggcgacggccggggccgcattcctgggggccgggcggtgctcccgcccgcctcgataaaaggctccggggccggcggcggcccacgagctacccggaggagcgggaggcgccaagctctaga

SEQ ID NO: 21 - DNA, 283 bp, Artificial Sequence
  This is the synthetic promoter for c6-16.
cggccgtcgccatatttgggtgtccgctctaaaaataactcccccaacacctgctgcctgccccgtcgccatatttgggtgtcggcaggcagcaggtggtggccaacacctgctgcctgccccaacacctgctgcctgccccgtcgccatatttggtgtccgccctcggccggggccgcattcctgggggccgggcggtgctcccgcccgcctcgataaaaggctccggggccggcggcggcccacgagctacccggaggagcgggaggcgccaagctctaga

SEQ ID NO: 22 - DNA, 263 bp, Artificial Sequence
  This is the snythetic promoter for c6-39.
cggccgtccgccctcgggggagttatttttagagcgccaacacctgctgcctgccccgtcgccatatttgggtgtcggcaggcagcaggtgttgggggagttatttttagagcgccgtcgccatatttgggtgtcccgagggcggacggccggggccgcattcctgggggccgggcggtgctcccgcccgcctcgataaaaggctccggggccggcggcggcccacgagctacccggaggagcgggaggcgccaagctctaga

SEQ ID NO: 23 - protein, 40 aa, Artificial Sequence
  This is a HV-growth hormone releasing hormone (“GHRH”) analog.
HVDAIFTNSYRKVLAQLSARKLLQDILNRQQGERNQEQGA

SEQ ID NO: 24 - protein, 40 aa, Artificial Sequence
  This is an amino-acid sequence for Pig-GHRH.
YADAIFTNSYRKVLGQLSARKLLQDIMSRQQGERNQEQGA

SEQ ID NO: 25 - protein, 40 aa, Artificial Sequence
  This is an amino-acid sequence for Bovine-GHRH.
YADAIFTNSYRKVLGQLSARKLLQDIMNRQQGERNQEQGA

SEQ ID NO: 26 - protein, 40 aa, Artificial Sequence
  This is an amino-acid sequence for Dog-GHRH.
YADAIFTNSYRKVLGQLSARKLLQDIMSRQQGERNREQGA

SEQ ID NO: 27 - protein, 40 aa, Artificial Sequence; identical to SEQ ID NO: 24
  This is an amino-acid sequence for Cat-GHRH.

SEQ ID NO: 28 - protein, 40 aa, Artificial Sequence
  This is a TI-growth hormone releasing hormone (“GHRH”) analog.
YIDAIFTNSYRKVLAQLSARKLLQDILNRQQGERNQEQGA

SEQ ID NO: 29 - protein, 40 aa, Artificial Sequence
  This is an amino-acid sequence for Ovine-GHRH.
YADAIFTNSYRKILGQLSARKLLQDIMNRQQGERNQEQGA

SEQ ID NO: 30 - protein, 43 aa, Artificial Sequence
  This is an amino-acid sequence for Chicken-GHRH.
HADGIFSKAYRKLLGQLSARNYLHSLMAKRVGSGLGDEAEPLS

SEQ ID NO: 31 - protein, 40 aa, Artificial Sequence
  This is an amino-acid sequence for Horse GHRH.
XADAIFTNNYRKVLGQLSARKILQDIMSRXXXXXXXXXXX

SEQ ID NO: 32 - protein, 40 aa, Artificial Sequence
  This is an amino-acid sequence for human (1-40) GHRH.
YADAIFTNSYRKVLGQLSARKLLQDIMSRQQGESNQERGA

SEQ ID NO: 33 - protein, 40 aa, Artificial Sequence
  This is a TV-growth hormone releasing hormone (“GHRH”) analog.
YVDAIFTNSYRKVLAQLSARKLLQDILNRQQGERNQEQGA

SEQ ID NO: 34 - protein, 40 aa, Artificial Sequence
  This is a TA-growth hormone releasing hormone (“GHRH”) analog.
YADAIFTNSYRKVLAQLSARKLLQDILNRQQGERNQEQGA

SEQ ID NO: 35 - protein, 44 aa, Artificial Sequence
  This is a human (1-44) growth hormone releasing hormone (“GHRH”).
YADAIFTNSYRKVLGQLSARKLLQDIMSRQQGESNQERGARARL

SEQ ID NO: 36 - protein, 153 aa, Artificial Sequence
  This is an IGF-I amino acid sequence.
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

(19) United States
(12) Patent Application Publication
(10) Pub. No.: US 2010/0247437 A1
(43) Pub. Date: Sep. 30, 2010

(54) MATERIALS AND METHODS FOR THE DELIVERY OF BIOMOLECULES TO CELLS OF AN ORGAN

(75) Inventor: Ruxandra Draghia-Akli, Brussels (BE)

Correspondence Address: VGX Pharmaceuticals, LLC, 1787 Sentry Parkway West, Building 18, Suite 400, Blue Bell, PA, 19422, US

(73) Assignee: VGX PHARMACEUTICALS, LLC, Blue Bell, PA (US)

(21) Appl. No.: 12/680,278

(22) PCT Filed: Sep. 24, 2008
(86) PCT No.: PCT/US08/77499

§ 371 Date: Mar. 29, 2010

Related U.S. Application Data
(60) Provisional application No. 60/976,660, filed on Oct. 1, 2007.

(51) Int. Cl. A61K 49/00; A61K 31/7105; A61K 31/7088; A61K 38/02; A61P 43/00; A61N 1/30

(52) U.S. Cl. 424/9.1; 514/44 A; 514/44 R; 514/1.1; 604/21

(57) ABSTRACT

The present invention is directed towards materials and methods of delivering biomolecules to cells that define an organ, and the organ being in situ, and electroporating cells defining the organ by delivering to the cells an amount of energy effective to electroporate the cells. The present invention is also directed towards materials and methods of expressing heterologous polypeptides in organs of a subject and electroporating cells defining the organ by delivering to the cells an amount of energy effective to electroporate the cells.

### BACKGROUND OF THE INVENTION

Disease and injury often inflicts a subject specifically in an area of an organ or concentrated in the organ. If the organ is vital, then the disease or injury can often become fatal. Many existing treatments for organ specific diseases and injuries are administered to subjects through systemic routes, which produce less predictable outcomes and can cause a multitude of adverse side effects. Many therapies that are delivered through systemic routes can cause substantial stress and harm to the liver and kidney for example, and thus, must be carefully examined before used to treat a patient. Furthermore, some treatments cause significant risk to the patient because they are highly invasive, especially when considering therapies that require direct access to the inflicted organ.

Recently, the delivery of specific genes to somatic tissue in a manner that can correct inborn or acquired deficiencies and imbalances was proved to be possible. Gene-based drug delivery offers a number of advantages over the administration of recombinant proteins. These advantages include the conservation of native protein structure, improved biological activity, avoidance of systemic toxicities, and avoidance of infectious and toxic impurities. In addition, gene therapy allows for prolonged exposure to the protein in the therapeutic range, because the protein is synthesized and secreted continuously into the circulation. The primary limitation of using recombinant protein is the limited availability of protein after each administration due to their relative short half-life in serum. Gene therapy using injectable DNA plasmid vectors overcomes this, because a single injection into the patient's skeletal muscle permits physiologic expression of the protein for extensive periods of time (WO 99/05300 and WO 01/06988). Injection of the vectors promotes the production of enzymes, cytokines, and/or hormones in animals in a manner that more closely mimics the natural process. Furthermore, among the non-viral techniques for gene transfer in vivo, the direct injection of plasmid DNA into muscle tissue is simple, inexpensive, and safe.

Direct plasmid DNA transfer is currently the basis of many emerging therapeutic strategies, as it avoids the potential problems associated with viral genes or lipid particles. Skeletal muscle is a preferred target tissue, because the muscle fiber has a long life span and can be transduced by circular DNA plasmids. Skeletal muscle borne plasmids have been expressed efficiently over months or years in immunocompetent hosts. Plasmid DNA constructs are attractive candidates for direct therapy into a subject's skeletal muscle because they are well-defined entities, which are biochemically stable and have been used successfully in the past. Previously, reports in mice have showed human GHRH cDNA delivered to muscle by an injectable myogenic expression vector transiently stimulated GH secretion over a period of two weeks. The injectable vector system was optimized by incorporating a strong synthetic muscle promoter coupled with a novel protease-resistant GHRH molecule that has a substantially longer half-life and greater GH secretory activity (pSP-HV-GHRH).

The ability to program recombinant gene expression in cardiac myocytes in vitro and in vivo is desirable because many inherited and acquired cardiovascular diseases exist. Cardiovascular gene therapy was developed as an approach to increase angiogenesis, promote myocardial cell division, and inhibit apoptosis; however, there are limits to such treatment of cardiac disease including the relatively low levels of expression that have been achieved using conventional gene delivery vectors and delivery systems.

Previous studies evaluating gene delivery strategies to the myocardium have identified a number of methodological obstacles related to vector delivery. For example, a single injection of adenovirus into the coronary artery limits myocardial transfection efficacy to less than 1% (Logeart et al., 2000). Transient downstream coronary artery occlusion before coronary injection increases delivery efficacy. Transfection efficiency is further increased to more than 5% when the arterial occlusion is combined with coronary sinus occlusion to minimize venous efflux (Logeart et al., 2001). However, this approach is impractical for clinical applications. Although intra-coronary infusion is associated with decrease myocardial inflammation or necrosis (Barr et al., 1994) and potentially increased efficacy, localization may be difficult in atherosclerotic, often occluded coronary vessels. Furthermore, other investigators showed poor reporter gene expression after intra-coronary infusion (Wright et al., 2001), and there is an inverse relationship between delivery into the myocardium and distance from arterial lumen (Wilensky et al., 1999). Viral vectors, in particular adenoviruses and adeno-associated have shown high transductive efficacy to the heart upon direct myocardial injection (Chu et al., 2003), nevertheless, epicardial approaches are limited by increased morbidity and mortality of open chests techniques.

There still remains a need for effective therapies based on direct application of a biomolecule, such as a pharmaceutical drug or vaccine or gene, into a diseased or injured organ. There remains a need for a system and/or methodology that enables delivery of such biomolecules to a specific site, such as a diseased or injured organ, that provides efficient delivery and safety.

### SUMMARY OF INVENTION

In an aspect of the present invention, provided are methods of delivering biomolecules to cells that define an organ, and the organ being in situ. The methods of delivering including delivering the biomolecules to the organ and providing an amount of energy to the organ effective to electroporate the cells that define the organ.

In another aspect of the present invention, provided are methods of expressing heterologous polypeptides in organs of a subject. The methods of expressing the heterologous polypeptides include injecting a needle transdermally, transthoracically, or directly to the organ to deliver an encoding nucleic acid molecule that encodes the heterologous polypeptide, and electroporating cells defining the organ by delivering to the cells an amount of energy effective to electroporate the cells.

In yet another aspect of the present invention, provided are systems adapted for the delivery of an encoding nucleic acid molecule to an organ of a mammal for the expression of a polypeptide from cells defining the organ. The systems comprise a needle for delivery of the encoding nucleic acid molecule to the organ, and an electorporation device capable of delivering pulses of energy at an intensity effective for electroporation of the cells of the organ.

### BRIEF DESCRIPTION OF DRAWINGS

The invention can be more fully understood from the following detailed description and the accompanying drawings and Sequence Listing which form a part of this application.

FIG. 1 shows fluorescent images of Sol8 cells transfected with GFP expressing plasmids under the control of synthetic promoters (Under 20× Magnification);

FIG. 2 shows a graph of circulating SEAP levels (pg/ml) over time after direct plasmid injection into the myocardium followed by electroporation; and

FIG. 3 shows a fluorescent image representing GFP expression after direct plasmid injection into the myocardium followed by electroporation.

FIG. 4 shows the design of muscle synthetic promoters elements in the constructs with the highest in vitro reporter gene activity compared with skeletal α-actin 448 promoter (“SK448”).

### DETAILED DESCRIPTION OF PREFERRED EMBODIMENTS

“Constant current” is used herein to mean a current that is received or experienced by a tissue, or cells defining said tissue, over the duration of an electrical pulse delivered to same tissue. The electrical pulse can be delivered from the electroporation devices described herein, and like devices known in the art. The constant current remains at a constant amperage in said tissue over the life of an electrical pulse because the electroporation device provided herein has a feedback element, preferably instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse.

The phrase, “amount of energy . . . effective to electroporate cells,” refers to an amount of energy delivered from an electrode to a desired cell, such as one that is part of an organ of interest. Preferably, the amount of energy is enough to electroporate the cells but below the amount that would kill the cells; therefore permitting the migration of biomolecules through the membrane of the cells.

“Expression construct” or “DNA expression construct” is used herein, interchangeably, to mean a nucleic acid sequence that contains at least a promoter, an encoding DNA sequence and a polyadenylation and/or a 3′ untranslated region sequence that are operatively linked, and allow the expression of the encoding DNA within a cell.

“Promoter” refers to a nucleic acid sequence at which the initiation of transcription occurs and transcription is regulated. Promoters are capable of controlling transcription of another nucleic acid fragment.

“Tissue-specific promoter” and “tissue-preferred promoter” are used interchangeably, and refer to a promoter that is drives expression of an operably linked encoding sequence predominantly, but not necessarily exclusively, in one tissue or organ.

“In situ” is used herein to refer to the natural location or position of the referred-to subject. With respect to the organs being treated by the present invention, in situ refers to the organs and underlying tissue/cells being in their natural location with the subject having such organ being alive.

“Biomolecule” is used herein to mean a molecule that is organic and useful in the application to a subject, preferably a mammal. The biomolecule is, for example, biomolecule is a polynucleic acid, polypeptide, or pharmaceutical compound, and preferably such biomolecules that are known to have therapeutic effect in an injured or diseased organ of a mammal.

The present invention relates to devices, biomolecules, and electroporation techniques disclosed herein, that when combined, enables delivery of biomolecules to an organ of a patient for a number of prophylaxes, treatments or therapies. In some aspects, the invention includes an electroporation device including electrodes that can be applied nearby the desired organ and biomolecules that can be delivered directly to cells of the desired organ for therapies. The biomolecules can be chosen from one or more of a polynucleic acid, polypeptide, or pharmaceutical compound, and preferably these biomolecules are those known to have therapeutic effect in an injured or diseased organ of a mammal. In embodiments where the biomolecule is a polynucleic acid, the polynucleic acid can be selected from one that is useful for gene therapy or DNA vaccination, among other therapeutic uses of polynucleic acids. Preferably, the polynucleic acid is a siRNA, linear DNA, or DNA expression construct.

In some embodiments, when the biomolecule is a DNA expression construct, the expression construct preferably includes an encoding sequence. The encoding sequence can be one that is native or non-native to the subject. In some embodiments, the encoding sequence encodes GHRH, IGF-1, GnRH, caspases, or other growth factors or hormones. Preferably, the encoding sequence encodes GHRH, IGF-1, or GnRH. In some embodiments, the DNA expression construct also comprises a promoter that is operably linked to the encoding sequence. The promoter can be a constituitive or tissue specific promoter that can effectively drive expression of the encoding sequence in the selected organ. Preferably, the promoter is a tissue specific promoter, and more preferably is selected from SPc5-12, SPc1-26, or SPc6-39.

In embodiments where the biomolecule is a polypeptide, the polypeptide can be a native polypeptide or a heterologous (or non-native) polypeptide, or fragments thereof including functional elements like binding elements, catalytic elements, or antigenic fragments. In some embodiments, the polypeptide can replace or supplement, in a subject, an absent or defective protein that is the result of some genetic disease or injury to an organ. In some embodiments, the polypeptide can replace or supplement, in a subject, a downregulation of a protein that is integral to a metabolic process.

The delivery of the biomolecules disclosed herein is contemplated for delivery into the subject by means that require the minimum amount of invasiveness and, ultimately, a reduction in the rate of mortality. The delivery methods can include those that utilize a long needle of a syringe or a catheter equipped with a delivery mechanism, all of which allow for delivery of the biomolecules to a live subject with minimized invasiveness. Preferably, the delivery methods are selected from either transdermal, transthoracic, or direct delivery in order to deliver the biomolecule to the organ. The term “transdermal” is used herein to mean passage through the dermal layer of skin to reach a desired organ. The term “transthoracic” is used herein to mean passage through and into the thoracic cavity to gain access to the heart of a subject. The term “direct delivery” is used to mean the delivery of a biomolecule directly to an organ that is accessible without passage through the skin, e.g., delivery to the skin or eye or gonad, or vascular delivery.

In some embodiments, the methods further include providing a pharmaceutical formulation of the biomolecule for delivery to a subject having the organ, and preparing a delivery apparatus with the pharmaceutical formulation. The step for providing an amount of energy can further comprise: situating at least one pair of electrodes around the organ, generating the electroporating amount of energy, and transmitting the electroporating amount of energy to the pair of electrodes

In one aspect of the present invention, the invention further includes imaging the placement of the biomolecule to the organ. The imaging can be provided by one of any available imaging machines, and includes the underlying methodologies of such machine, which are available to physicians in a clinic office or surgery room.

In one aspect of the present invention, methods are provided for delivering a biomolecule to a cell that defines an organ, the organ being in situ, comprising: delivering the biomolecule to the organ, and providing an amount of energy to the organ effective to electroporate the cells that define the organ.

In yet another aspect of the present invention, provided are systems adapted for the delivery of an encoding nucleic acid molecule to an organ of a mammal for the expression of a polypeptide from cells defining the organ. The systems comprise a needle for delivery of the encoding nucleic acid molecule to the organ, and an electorporation device capable of delivering pulses of energy at an intensity effective for electroporation of the cells of the organ. The electroporation device can be selected from among the many electorporation devices adapted for delivering an electrical current to a patient, e.g., a mammal, and preferably the electroporation device is a constant current electroporation device. In some examples, the electroporation device comprises a defibrillator, preferably a Physio-Control Lifepak 6s defibrillator. In some embodiments, the systems further comprise an imaging machine that is capable of detecting the placement of the needle in the mammal. The imaging machine can be selected from any of a number of commercially available imaging machines such as an ultrasound device, magnetic resonance imaging, x-ray, radio-scan, or other non-invasive needle detection devices. Preferably, the imaging machine is an ultrasound device.

### DNA Expression Constructs

In some embodiments, the biomolecule intended to be delivered to a subject, preferably a mammal, is a DNA expression construct. The expression constructs may be administered to a subject, and preferably, the DNA expression constructs are administered directly to an organ located within the subject. Preferably, the administration is done with the subject alive and with minimal amount of invasive procedures—all to enhance the likelihood of survival of the subject after undergoing administration of the biomolecule.

The DNA expression construct can have a variety of functional nucleic acid sequences. In some instances, the DNA expression construct includes an encoding sequence, which in many embodiments is a heterologous gene, and a promoter driving the expression of the encoding gene. The promoter can be chosen from one of a number of known promoters for expression in eukaryotic cells. Preferably, the promoter is tissue specific, and in some instances, the promoter is a synthetic promoter. In some embodiments, the tissue specific promoter is synthetic and chosen from either Spc5-12, Spc1-26, or Spc6-39.

Promoters

A promoter is a control sequence that is a region of a nucleic acid sequence at which the initiation and rate of transcription are controlled. The promoter can contain genetic elements where regulatory proteins and molecules may bind such as RNA polymerase and transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one of naturally-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including polymerase chain reaction amplification. Furthermore, it is contemplated the control sequences that direct transcription and/or expression of sequences within non-nuclear organelles such as mitochondria, chloroplasts, and the like, can be employed as well.

In some embodiments, a promoter and/or enhancer employed is one that effectively directs the expression of the encoding sequence in the cell type, organelle, and organism chosen for expression. Those of skill in the art of molecular biology generally know the use of promoters, enhancers, and cell type combinations for protein expression. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. In a specific embodiment the promoter is a synthetic myogenic promoter.

The identity of tissue-specific promoters or elements, as well as assays to characterize their activity, is well known to those of skill in the art. Examples of such regions include the human LIMK2 gene, the somatostatin receptor 2 gene, murine epididymal retinoic acid-binding gene, human CD4, mouse alpha2 (XI) collagen, DIA dopamine receptor gene, insulin-like growth factor II, human platelet endothelial cell adhesion molecule-1.

In some embodiments, cardiac specific-synthetic promoters are contemplated. Some examples of cardiac specific-synthetic promoters include synthetic promoters (“SP”): c5-12 (SEQ ID NO.: 5). Other specific embodiment utilizes other cardiac specific synthetic promoters such as c1-26 (SEQ ID NO.: 16); c2-26 (SEQ ID NO.: 17); c2-27 (SEQ ID NO.: 18); c5-5 (SEQ ID NO.: 19); c6-5 (SEQ ID NO.: 20); c6-16 (SEQ ID NO.: 21); or c6-c39 (SEQ ID NO.: 22). The cardiac-specific synthetic promoters drive transcriptional activity of the expressible gene in a population of cells that is higher than the transcriptional activity of the expressible gene driven by a control-promoter in the same population of cells. In some embodiments, the cardiac-specific-synthetic promoters comprise a first-combination of cis-acting regulatory elements. The cis-acting regulatory elements can be utilized in addition to the cardiac-specific synthetic promoters to further enhance expression and can include: SRE (SEQ ID NO.: 1); MEF-1 (SEQ ID NO.: 2); MEF-2 (SEQ ID NO.: 3); and TEF-1 (SEQ ID NO.: 4).

In some embodiments, provided are methods for using cardiac specific-synthetic DNA expression constructs for expressing a gene in a cardiac cell. The method comprises delivering into the cardiac cell a cardiac specific-synthetic DNA expression construct. The cardiac-specific-synthetic DNA expression constructs comprise a cardiac-specific-synthetic-promoter operatively-linked to an expressible gene.

Certain embodiments describe the expressible-gene comprising a nucleic acid sequence that encodes a growth-hormone-releasing-hormone (“GHRH”) or functional biological equivalent thereof. The encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide. In another specific embodiment, the encoded GHRH or functional biological equivalent thereof is of formula (SEQ ID NO.: 6): The cardiac specific-synthetic DNA expression constructs of this invention also comprises SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, SEQ ID NO.: 14, or SEQ ID NO.: 15.

Initiation Signals and Internal Ribosome Binding Sites.

A specific initiation signal also may be required for efficient translation (synthesis of the encoded protein) of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, internal ribosome entry sites (“IRES”) elements are used to create multigene, or polycistronicmessages. IRES elements are able to bypass the ribosome scanning model of 5′ methylated Cap-dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Multiple Cloning Sites.

Vectors can include a multiple cloning site (“MCS”), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing Sites.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression.

Polyadenylation Signals.

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the bovine or human growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

Origins of Replication.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

Selectable and Screenable Markers.

In certain embodiments of the invention, the cells that contain the nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker, such as the antibiotic resistance gene on the plasmid constructs (such as kanamycin, ampicillin, gentamycin, tetracycline, or chloramphenicol).

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as green fluorescent protein (“GFP”), whose basis is colorimetric analysis, are also contemplated. Alternatively, screenable enzymes may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

### Delivery of Biomolecules

Any one of available delivery methods can be utilized to deliver the biomolecule to the organ. The delivery method is selected to enable delivery of biomolecules, preferably DNA expression constructs, to the organ of a subject, preferably a mammal, so that the subject experiences minimal injury and minimal risk of fatality. The cells that form the organ are in contact with the biomolecule, often as a part of a pharmaceutical or delivery formulation.

In some embodiments, the method of delivery of the biomolecule includes a needle, e.g., a syringe, which can hold the biomolecule and deliver same biomolecule to the organ.

In one example, a mammal can be injected directly into the apex of the heart using a transthoracic technique under the ultrasound visualization of the delivery needle. More particularly, the injections can be performed using sterile Terumo Spinal Needles −25G, 3^{1/2}″ (Burns Veterinary Supplies, SN2590). The specific needle can be replaced with an alternate needle having similar properties but varied size or depth in order to more efficiently deliver biomolecules to the patient of interest, for example at the site of another organ, including the lung, liver, kidney, gonad, and others. An appropriate injection site is determined by finding a site in which all major surface blood vessels can be avoided during delivery.

In another embodiment, the biomolecule can be delivered via a catheter and balloon technique. A catheter can be inserted in one of the main arteries or veins of the target organ. The delivery region would be temporarily isolated from the blood flow by a balloon that is inserted into the said blood vessel, and then inflated, to allow the biomolecule formulation to be in contact with the organ cells during the electroporation procedure. After electroporation, the balloon can be deflated and the region normally vascularized.

In some embodiments the biomolecule is a nucleic acid sequence. In one example the nucleic acid sequence is DNA sequence, for example, a plasmid or DNA expression construct. The amount of DNA that can be delivered using the techniques provided herein for delivery into an organ is from about 0.01 μg to about 1.0 g. In some embodiments, the amount of DNA delivered to an organ is from about 0.01 μg to about 500 μg; from about 0.01 μg to about 100 μg; from about 0.01 μg to about 10 μg; from about 0.01 μg to about 1.0 μg; from about 0.1 μg to about 500 μg; from about 0.1 μg to about 100 μg; from about 0.1 μg to about 10 μg; from about 0.1 μg to about 1.0 μg; from about 1.0 μg to about 500 μg; from about 1.0 μg to about 100 μg; or from about 1.0 μg to about 10 μg.

The encoding DNA sequence contained in the DNA expression construct can be selected from any desired gene or synthetic encoding sequence that relates to a disease of interest that afflicts the target organ for treatment. In some embodiments, the encoding DNA sequence can be selected from growth hormone releasing hormone (GHRH), insulin growth factor-1 (IGF-1), caspases, and other growth factors enzymes, cytokines, or hormones.

In some embodiments, the DNA expression construct includes an encoding DNA sequence that encodes GHRH, including functional analogs thereof. In some embodiments the encoding sequence encodes HV-GHRH (SEQ ID NO.: 23), pig-GHRH (SEQ ID NO.: 24), bovine-GHRH (SEQ ID NO.: 25), dog-GHRH (SEQ ID NO.: 26), cat-GHRH (SEQ ID NO.: 27), TI-GHRH (SEQ ID NO.: 28), ovine-GHRH (SEQ ID NO.: 29), chicken-GHRH (SEQ ID NO.: 30), horse-GHRH (SEQ ID NO.: 31), human (1-40)-GHRH (SEQ ID NO.: 32), TV-GHRH (SEQ ID NO.: 33), TA-GHRH (SEQ ID NO.: 34), human (1-44)-GHRH (SEQ ID NO.: 35), or GHRH (1-40) OH (SEQ ID NO.: 6).

In some embodiments, the DNA expression construct includes an encoding DNA sequence that encodes IGF-I, including functional analogs thereof. In some embodiments the encoding sequence encodes IGF-I (SEQ ID NO.: 36) and analogs thereof that retains substantially similar biological function.

Embodiments of the present invention also include, although not explicitly denoted, DNA expression constructs that include the different elements of the construct, including the promoter, enhancer, and encoding sequence described herein, arranged in a variety of different combinations. The resulting DNA expression construct can be tested in vitro to determine whether the construct is able to express the encoding sequence in the cell.

The molecule can be delivered in any one of available formulation suitable for delivery to a subject, such as mammals, including humans and non-human primates. In some embodiments, the biomolecule is formed into a pharmaceutical formulation for the biomolecule to be delivered to a subject. The biomolecule is preferably housed in a delivery apparatus along with a pharmaceutical formulation. The pharmaceutical formulations comprises said biomolecule and a pharmaceutically acceptable carriers, diluents, fillers, salts and other materials well-known in the art depending upon the dosage form utilized and the method of delivery/administration.

### Electroporation System

Aspects of the present invention relate to—after introducing or inserting molecules to the organ—providing of an amount of energy to the organ effective to electroporate cells contained therein. In some embodiments, the process of providing an amount of energy step further comprises: situating at least one pair of electrodes around the organ, generating the electroporating amount of energy, and transmitting the electroporating amount of energy to the pair of electrodes.

In a number of instances, there is an additional step in the process of delivering molecules to cells of an organ, which step can include imaging the placement of the molecule to the organ. In a preferred embodiment, the imaging of the placement of the molecule to the organ includes using an ultrasound device to visualize the placement of the molecules, such as within a needle or syringe. Any commercially available ultrasound device can be utilized with the remaining aspects of the present invention in order to efficiently deliver biomolecules to the organ, such as the heart.

In an embodiment of the electroporation device (“EP Device”), the EP Device is a constant current EP Device and includes the following major functional elements, each element being described in terms of its hardware functionality. The central element of the EP Device is the controller, which is responsible for controlling various peripheral devices connected to it. The controller is responsible for: (1) Performing impedance testing to determine if electroporation should be performed; (2) Generating the electroporation firing sequence for the electrode assembly by controlling the current waveform generator; (3) Sensing and processing user commands; (4) Providing the user with status information; (5) Saving and retrieving electroporation data (e.g. voltage and current curves) to and from memory; and (6) Transmitting electroporation data to a PDA device or personal computer via the communications port.

Another method for delivering electric pulses to an organ in a subject, preferably the heart, is delivery of electrical pulses using an electrical defibrillation. The electrical pulse can be applied to the heart of a subject for example. In one example with application to the heart, the electrical pulse can be delivered within a short time after onset of a pathological condition and biomolecule delivery. One way of providing electrical pulses with electrical defibrillation is the use of an external defibrillator. External defibrillators send electrical pulses to the heart of a subject through electrodes applied to the torso of the subject. External defibrillators are known to a skilled artisan and can be found and used in hospital emergency rooms, operating rooms, and in emergency medical vehicles.

Another component of the EP Device is the current waveform generator. The current waveform generator generates a current pulse train waveform that passes through the electrodes of the electrode array in accordance with a programmed sequence. The pulse train is square in shape and the number of pulses is limited by software. One pulse is applied to each electrode set. Typically, each pulse is between 1 μs and 500 ms in duration and occurs at a rate of 1 Hz. In some embodiments, the EP Device can deliver a pulse between 1 μs and 100 ms; between 1 μs and 50 ms; between 1 μs and 10 ms; between 1 μs and 1 ms; between 1 μs and 500 μs; between 1 μs and 100 μs; between 1 μs and 50 μs; between 100 μs and 500 ms; between 100 μs and 100 ms; between 100 μs and 50 ms; between 100 μs and 10 ms; between 100 μs and 1 ms; between 1 ms and 500 ms; between 1 ms and 100 ms; between 1 ms and 50 ms; or between 10 ms and 100 ms. In some embodiments the pulse is about 10 μs, or about 50 ms, or in some examples 52 ms. The amplitude of the pulse train is programmable by the operator and ranges from 0.1 Amp to 2.0 Amp in increments of 0.1 Amp.

An additional component of the EP Device is the impedance tester. The impedance tester determines if the resistance of the load (e.g. muscle tissue) is sufficiently low. If the resistance is too high, the resulting voltage across the electrodes might be too high and cause heating and cell damage. Electroporation treatment may therefore be preceded by an impedance test. If any of the impedance measurements exceeds 100±xceeds 100he impedance measurementse test.g voltage across the electrodes might b The impedance test is an operator programmable feature, controlled by software that may be disabled during the operation.

In another embodiment, the impedance is measured with the help of a monitoring adapter. A monitoring adapter is provided that communicates a patient parameter to a medical device which enables detection of the monitoring adapter, when the adapter is connected to the medical device. As a result of detection of the adapter, operation of the medical device can be changed. Detection of the monitoring adapter is enabled based on the interface impedance of the monitoring adapter measured by the medical device. The medical device can be a defibrillator and the monitoring adapter can communicate with the defibrillator using a set of EKG monitoring electrodes. In one specific embodiment, the monitoring adapter is formed integral with the monitoring electrode pads. Alternatively, the monitoring adapter can be configured so that it is removably connected to electrode pads. A fault detector may also be provided within the monitoring adapter to detect a fault condition. The monitoring electrode system can include from 2-12 electrodes.

The impedance tester also functions as a safety feature in the EP Device in order to make it a safe device to operate. It indicates whether all of the electrodes have penetrated the same tissue and a circuit can be established. Electrodes in contact with air, especially dry air, have an extremely high resistance. If electroporation starts and one or more electrodes have not penetrated the tissue, the resulting electrode voltages can be thousands of volts, which might have lethal consequences and also damage the EP Device. For this reason, overload voltage protection may be implemented to prevent excessive voltages on the electrodes. For example, regardless of the electrical load (e.g. air or muscle tissue), the over-voltage protection may be engaged if Vᵢⱼ exceeds 200V for a period of no more than 1 ms. Vᵢⱼ is the voltage across electrode i and j where i,j=1 to 5. If the over-voltage protection engages, Vᵢⱼ goes to approximately 0 V until the next electroporation pulse is fired. While the EP Device is in the off state, the voltage across any electrode pair preferably does not exceed 10V.

A further component of the EP Device is a waveform logger. The waveform logger records electroporation voltage and current waveforms, which are to be continuously sampled during electroporation treatment. By sampling and monitoring the parameters of the electroporation procedure, an operator can more easily analyze possible problems and adjust the settings in the event that an electroporation procedure fails or doesn't achieve desirable results. An exemplary sample rate is 2000 samples per second, about 104 samples for each of the 5 current pulses. An exemplary total sample period is 4152 ms with sampling starting approximately 50 ms before the first pulse is fired and stopping about 50 ms following the last pulse. The voltage and current waveforms may be quantified into a 12-bit digital representation with ±1 least significant bit (“LSB”) linearity. The current waveform resolution should preferably be at least 5 mA and the voltage waveform resolution should preferably be at least 0.25 V.

The EP Device can include other elements such as a means for displaying or otherwise notifying the operator as to the status of the system. These means may include an information display panel, such as an LCD. The LCD is preferably of the character display type and is preferably capable of displaying 4 lines of 20 characters each. The LCD is also preferably equipped with a back-light that can be switched on and off by means of a toggle switch. Status information may also be provided by means of a buzzer sounding at various pitches. Each pitch preferably has a different meaning, as controlled by the software. For example, the volume of the buzzer may have 3 programmable settings and range roughly from 60 to 80 dB at a distance of 1 meter from the buzzer. The sound pressure level range is only given as reference. The sound level is deemed acceptable if it is audible in a noisy environment (e.g. a farm) if set to its highest level and it is not too loud in a quite environment (e.g. an office) if set to its lowest level. In addition, the EP Device may be equipped with an LED to designate whether the unit is turned on or off.

Another optional component of the EP Device is a means for entering operating parameters. For example, the EP Device operating parameters may be programmed by an operator via a numeric keypad. In a preferred embodiment, the numbers input into the keypad are displayed on the LCD. Typical parameters that can be programmed are the electroporation pulse current, impedance test enable/disable, and electroporation firing delay. The features related to the keypad are also controlled by software.

A further component of the EP Device is a communications port that can be used to upload electroporation waveform data to another device, such as a PDA or PC, for viewing purposes. Preferably, the communications port is an optical serial communications port, such as an infrared (“IR”) port.

The EP Device may also possess a memory component. The memory component stores electroporation waveform data and operating parameters. Preferably, the memory is nonvolatile, meaning it retains its data even if the EP Device is off. To conserve memory, electroporation waveform data may only be saved to memory during the active periods of the electroporation pulse train. During the inactive periods, sampled data may only be stored to memory if either one of the waveforms exceeds a specified threshold. For example, these thresholds may be a voltage threshold of 2 V and a current threshold of 10 mA. Data stored to memory during the inactive periods of the current pulse train may be time stamped so that the time index of the data is known once the waveforms are reconstructed. Provision may be made for the storage of up to 40 samples (20 ms) of data that occur during the inactive periods of the pulse train. Storage can be limited to 20 ms because software can specify that the remainder of the electroporation sequence will be aborted if anyone of the thresholds is exceeded for a cumulative period of more than 20 ms. An electroporation waveform data set requires about 2 kB of memory when the above compression technique is implemented. The EP Device preferably contains sufficient memory to save at least 600 electroporation waveform data sets.

Further components of the EP Device are a power source and a power switch. The power source is preferably a battery and is responsible for providing power to each of the EP Device's circuits. These circuits include a low voltage/low power capacity power supply for the controller and its peripherals, a low voltage and low power capacity power supply for the impedance tester, and a high power capacity power supply for the current waveform generator. The power-switch controls power to the EP Device and can be either on or off. In the off position, all electrical connections to electrode assembly are electrically neutral within 5 seconds after power is turned off.

### Electrodes or Energy Applicators

Electroporation of tissue, and thus cells, in a subject can be accomplished by delivering energy to the tissue using an applicator that makes contact with the subject. Applicators for delivering energy include needle electrodes, adhesive lead electrodes, and pads of a defibrillator

In some embodiments, the electrodes are catheter electrodes. Catheter electrodes are selected among those known to one of ordinary skill, including catheter electrodes used for tissue ablation or for internal defibrillators. As an example of a catheter electrode that can be modified to deliver an electroporating amount of energy is that described in U.S. Pat. No. 6,312,408, where a high level of energy is required to ablate tissue (which is not desirable in the present invention).

Energy—Pulse

A pulse that is constant-current can be utilized to achieve electroporation in the desired organ. The constant-current is a current that can be prevented from attaining an amplitude at which the cells are destroyed. For example a 50 mS pulse in a constant-current system has been shown to result in no net increase in Amperes in the porcine muscle (FIG. 8B of U.S. Pat. No. 7,245,963). Accordingly there is no net increase in heat, which assures cellular survival. Pulsing cannot alter the current because the current is preset at a level where cell death does not occur.

In one embodiment, the EP Device produces current pulses using a defibrillator, in one example a Physio-Control LifePak 6s defibrillator, at a total energy level of between 1 Joules and 1000 Joules, between 10 Joules and 500 Joules, between 50 Joules and 250 Joules, between 75 Joules and 150 Joules, or about 100 Joules, per standard defibrillation conditions.

Organ Diseases

Heart

Direct administration of a therapy to the heart is desirable for many diseases that afflict the heart. In some embodiments, biomolecules can be delivered using the devices and techniques provided herein to treat one or more of the following diseases or injuries: post-myocardial infarction, cardiomyopathies, storage diseases with cardiac involvement, or other conditions indicated mainly to the heart.

Lung

Direct administration of a therapy to the lung is desirable for many diseases that afflict the lung. In some embodiments, biomolecules can be delivered using the devices and techniques provided herein to treat one or more of the following diseases or injuries: physical trauma, emphysema, lung cancer, bronchitis, and other known injuries specific to the lung, as well as genetic diseases such as cystic fibrosis.

Liver

Direct administration of a therapy to the liver is desirable for many diseases that afflict the liver. In some embodiments, biomolecules can be delivered using the devices and techniques provided herein to treat one or more of the following diseases or injuries: cirrhosis, liver cancer, hepatitis, and other known injuries specific to the liver, as well as genetic diseases that have primary or secondary involvement of the liver, such as thalassemias, lysosomal or microsomal diseases, errors of metabolism, disorders of glycosylation, etc.

Kidney

Direct administration of a therapy to the kidney is desirable for many diseases that afflict the kidney. In some embodiments, biomolecules can be delivered using the devices and techniques provided herein to treat one or more of the following diseases or injuries, such as glomerulosclerosis, glomerulonefritis, primary or secondary genetic diseases with kidney involvement, secondary complications from heart disease or diabetes, etc.

Gonads

Direct administration of a therapy to the gonad is desirable for many diseases that afflict the gonad. In some embodiments, biomolecules can be delivered using the devices and techniques provided herein to treat one or more of the following diseases or injuries: such as cancer or infertility.

In embodiments where therapies or the DNA expression constructs are delivered to an organ other than the heart, the constructs will have replaced the muscle specific promoters with an appropriate tissue specific promoter (e.g., liver specific promoter for treatment of liver) or a constitutive promoter such as a CMV promoter.

Expression in Heart

Utilizing the materials and methods described herein, desired DNA including those expressing desired polypeptides can be delivered directly to the heart. By directly injecting into the heart, the delivered DNA can effectively treat trauma and disease in the heart, e.g., myocardial infarction.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Construction of Plasmids Driven by Tissue Specific Promoters

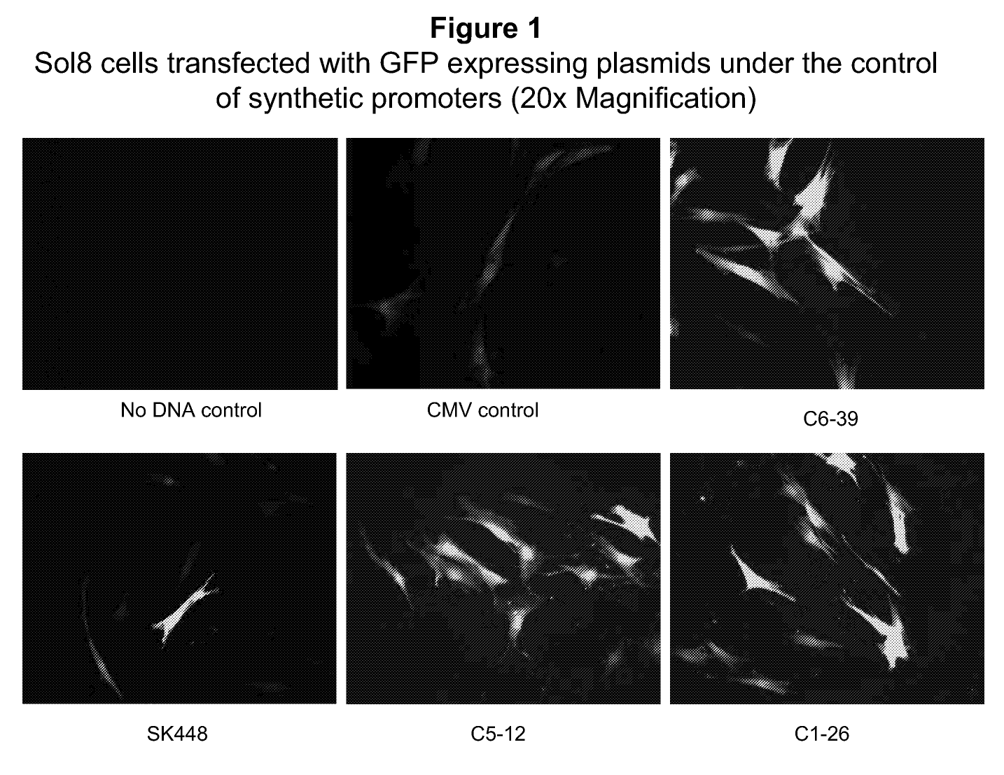

A short core fragment (i.e. “SK144”) of the chicken skeletal α-actin promoter (known as a very strong promoter) was used as the minimal sequence to randomly insert synthetic regulatory elements (Lee et al., 1994), (Chow et al., 1991). A description of the resulting synthetic promoters (“SP”) highly specific for muscle and/or cardiac cell expression has been published (US Patent Application No. 20040175727, “'727”). Briefly, the core motif of each regulatory element used in producing the SP was flanked by adjacent sequences that are conserved in the natural genes to allow the regulatory elements to anneal on the same face of the DNA helix. For example the serum regulatory element (“SRE”) sequence corresponds to the proximal SK SRE1, GCTGC motif adjacent to the MEF-1 is conserved in the muscle creatine kinase gene and rat myosin light chain gene. Different combinations of SRE, MEF-1, MEF-2 and TEF-1 oligonucleotide (FIG. 1, '727) were annealed and then capped by ligation with Sp1 elements, since Sp1 has been shown to act in synergy with SREs and E-boxes. It has also been shown that Sp1 binding sites are essential for de novo methylation protection of CpG islands and non-island DNA regions (Machon et al., 1998). Synthetic promoter libraries were generated from DNA fragments containing about 5-20 regulatory elements and ligated into a minimal actin-reporter plasmid that expresses the luciferase reporter gene.

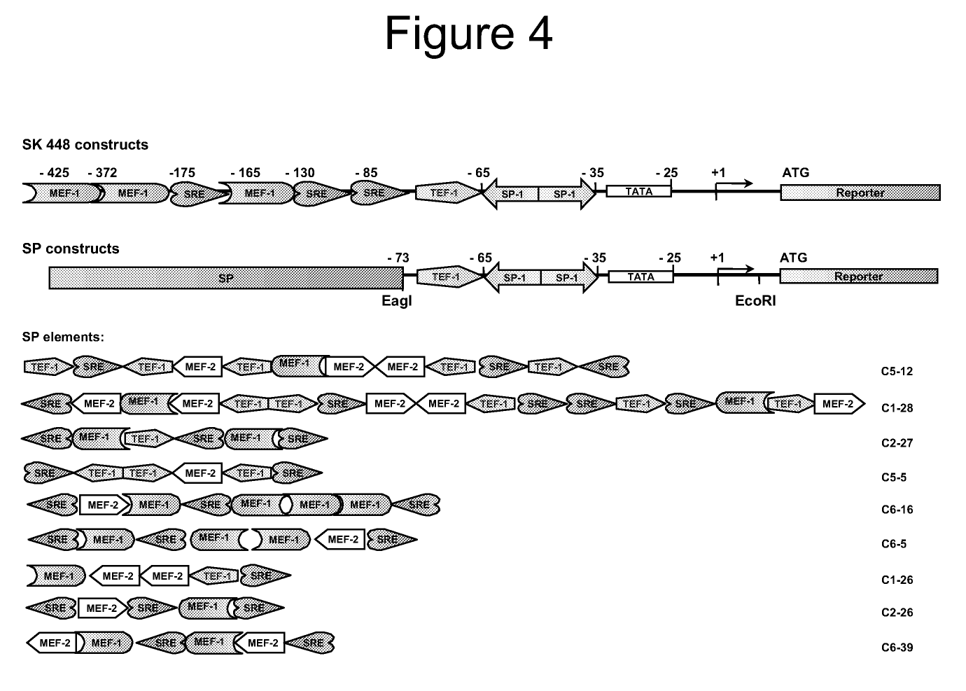

Screening synthetic promoters with high transcriptional activity. The in vitro luciferase activity was measured in more than 1000 different clones in 96 well dishes containing transiently transfected chicken primary myoblasts to determine the strength of the newly constructed synthetic promoters. A 448 bp promoter fragment (−424/+24) (“SK448”) of the avian skeletal α-actin gene was used as a specific expression control in cardiac and skeletal muscle (FIG. 4). The SK448 promoter control has been shown to be active in differentiated skeletal muscle cells, but not in myoblasts (Bergsma et al., 1986; Chow and Schwartz, 1990; Lee et al., 1994). Cytomegalovirus (“CMV”) basic promoter was also used as a ubiquitous promoter control. Newly generated synthetic promoters, CMV promoters, and SK448 promoters were inserted into reporter construct plasmids and transfected into cells then placed into differentiation media for up to 72 hours to initiate withdrawal from the cell cycle and to induce post-fusion differentiation and muscle-specific promoter activation. At the end of this period the cells were harvested and assayed for the reporter gene activity.

Promoters consisting of only multimerized single elements such as SREs, E-boxes, MEF-2 or TEF-1 had activities several-fold lower than the skeletal α-actin promoter 448. Clones that displayed transcriptional activity greater than 2 times that of SK448 activity were examined further. The results of these studies have been previously published (Li et al., 1999).

### Example 2

### In Vitro Activity of Sp-Driven Constructs into the Cardiac Tissue

For these experiments, pairs of plasmids were used (SK448, SPc1-26, SPc5-12 and SPc6-39): one plasmid in the pair expressed green fluorescent protein GFP, used for visualization of plasmid, the other expressed the secreted embryonic alkaline phosphatase (SEAP), to determine the level of transgene product expression and secretion from the pig cardiac muscle.

pSPx-xx-GFP is a series of plasmids for GFP expression. The GFP plasmids contain one of the tissue-specific, synthetic promoters described above (Li et al., 1999), which drive GFP expression. A GFP plasmid that includes a CMV promoter, pEGFP-N1 (Clontech, Mountain View, Calif.), was used as an in vitro positive control, and used to clone in the SPs. pEGFP-N1 encodes the GFPmut1 variant which contains the double-amino-acid substitution of Phe-64 to Leu and Ser-65 to Thr. The coding sequence of the EGFP gene contains more than 190 silent base changes which correspond to human codon-usage preferences. Sequences flanking EGFP have been converted to a Kozak consensus translation initiation site to further increase the translation efficiency in eukaryotic cells. SV40 polyadenylation signals downstream of the EGFP gene direct proper processing of the 3′ end of the EGFP mRNA. The vector backbone also contains an SV40 origin for replication in mammalian cells expressing the SV40 T-antigen. A neomycin-resistance cassette (neo^{r}), consisting of the SV40 early promoter, the neomycin kanamycin resistance gene of Tn5, and polyadenylation signals from the Herpes simplex thymidine kinase gene, allows stably transfected eukaryotic cells to be selected using G418 (Clontech Laboratories, Mountain View, Calif.)(G418 is a product used to select and maintain stable eukaryotic cell lines that have been transfected with vectors containing the gene for neomycin resistance) A bacterial promoter upstream of this cassette expresses kanamycin resistance in E. coli. The pEGFP-N1 backbone also provides a pUC19 origin of replication for propagation in E. coli and an f1 origin for single-stranded DNA production.

The plasmids were first tested in vitro. The purpose of this experiment was to screen the activity of GFP expression in myogenic cells (Sol8 cells) with plasmids driven by the different synthetic promoters of interest. (FIG. 1), while a SK448 driven plasmid was used as a tissue specific control. As shown, results indicate that the SP-driven plasmids transfected into myogenic cells result in high levels of GFP expression, compared to both ubiquitous promoter CMV and the naturally occurring tissue specific SK448.

pSPx-xx-SEAP is a series of plasmids for SEAP expression. The SEAP plasmids contain one of the tissue-specific, synthetic promoters described above (Li et al., 1999), which drive SEAP expression. The 3′ ends of SEAP transcripts are defined by the SV40 late poly(A) signal. The plasmids were constructed by inserting a 394 by Acc65I-HindIII fragment, containing the specific synthetic promoter sequences, between the Acc65I and HindIII sites of pSEAP-2 Basic Vector (Clontech Laboratories, Inc., Palo Alto, Calif.). The specific plasmid encoding for SEAP was delivered to the cardiac muscle of pigs and SEAP levels were measured in serum. SEAP levels have been previously used in vitro (Durocher et al., 2000), in mice (Nicol et al., 2002) and in both dogs and pigs (Draghia-Akli et al., 2002) as a reporter to investigate the conditions required for expression of secreted proteins. Although the SEAP protein is immunogenic in pigs, the immune-mediated clearance of the protein does not occur until 10 to 14 days after plasmid delivery. Thus, the levels of SEAP expression over a 2-week period can be analyzed and interpreted as a reliable measure of gene expression following plasmid transfer to the myocardium.

### Example 3

### In Vivo Delivery and Activity of Sp-Driven Constructs into the Cardiac Tissue of Pigs

In vivo expression of SP promoters was compared to that of the muscle specific SK448 promoter, after direct intra-cardiac injection in adult pig myocardium. For these experiments, pairs of plasmids were used (SK448, SPc1-26, SPc5-12 and SPc6-39): one plasmid in the pair expressed green fluorescent protein GFP, used for visualization of plasmid, the other expressed the secreted embryonic alkaline phosphatase (SEAP), to determine the level of transgene product expression and secretion from the pig cardiac muscle.

### Optimized Plasmids for the Comparison of Synthetic Promoters Driving the Expression of a GFP or SEAP.

Young hybrid pigs of mixed gender, 3 to 6 weeks of age, with weights between 15 and 40 kg, were used (n=4/group). Animals were group housed in pens at the Children's Nutrition Research Center, at Baylor College of Medicine, Houston, Tex., with ad libitum access to 24% protein diet (Producers Cooperative Association, Bryan, Tex.) and water. Plasmids were obtained using a commercially available kit (Qiagen Inc., Chatsworth, Calif., USA). Endotoxin levels were at less than 0.01 EU/μg, as measured by Kinetic Chromagenic LAL (Endosafe, Charleston, S.C.). Plasmid preparations were diluted in sterile water and formulated 1% weight/weight with poly-L-glutamate sodium salt (MW=10.5 kDa average) (Sigma, St. Louis, Mo.).

On Day 0 of the experiment, the animals were anesthetized with isofluorane (5% induction, 2-3% maintenance). The animals were placed on their backs, and their cardiac monitoring was performed by ultrasound (Aloka 500V, Corometrics Medical Systems, Inc.). The ultrasound was facilitated by directly applying on the skin around the chest Sonotrack ultrasound transmission gel of medium/low viscosity transmission (EchoSound, Reddsville, Pa.).

Injections and Electroporation. All animals were injected directly into the apex of the heart, into the myocardium using a transthoracic technique, under the ultrasound visualization of the delivery needle. Each mixture formulation contained 1 mg of each pair of plasmid (for instance, 1 mg pSPc6-39-SEAP and 1 mg pSPc6-39-GFP), in a total volume of 350 μL. The injections were performed using sterile Terumo Spinal Needles −25G, 3^{1/2}″ (Burns Veterinary Supplies, SN2590). All major surface blood vessels were avoided when finding an appropriate injection site. At the pre-determined time interval after plasmid injection, electroporation was initiated. The electroporation was performed using a Physio-Control LifePak 6s defibrillator, at a total energy level of 100 Joules, per standard defibrillation conditions. Animals recovered within a few minutes after the end of the procedure. No animal died during or after the procedure. All animals were killed at 10 days post-injection, and hearts collected for histological examination. Animals were maintained in accordance with NIH, USDA and Animal Welfare Act guidelines.

Blood collection. On days 0, 3, 7, and 10 of each experiment, animals were weighed at 8:30 AM and blood was collected by jugular vein puncture into Microtainer serum separator tubes. Blood was allowed to clot for 10 to 15 min at room temperature and subsequently centrifuged at 3000×g for 10 min and the serum stored at −80° C. until further analysis.

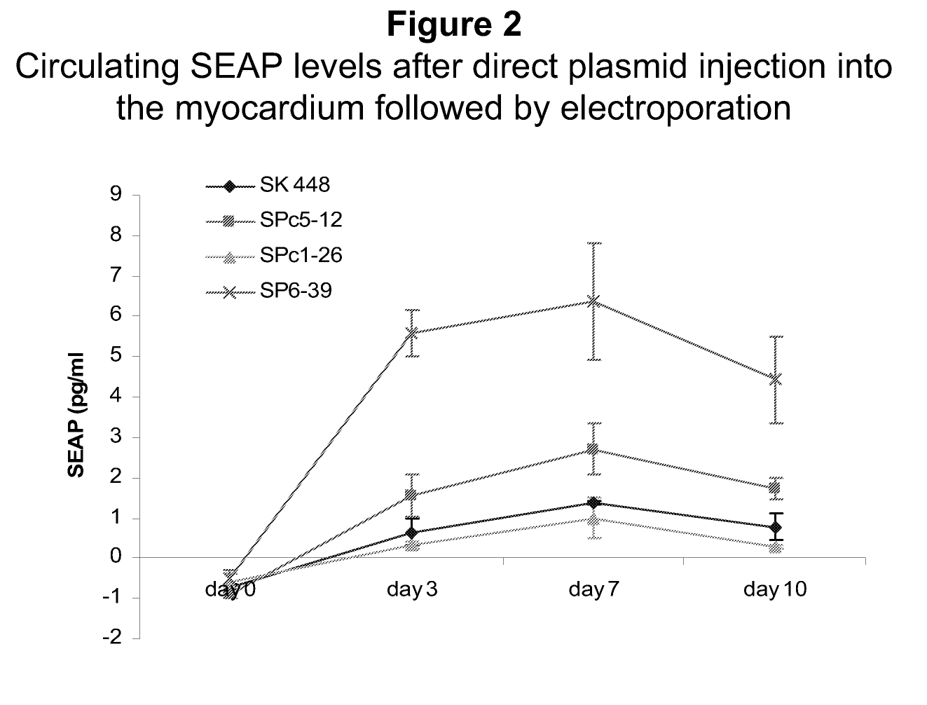

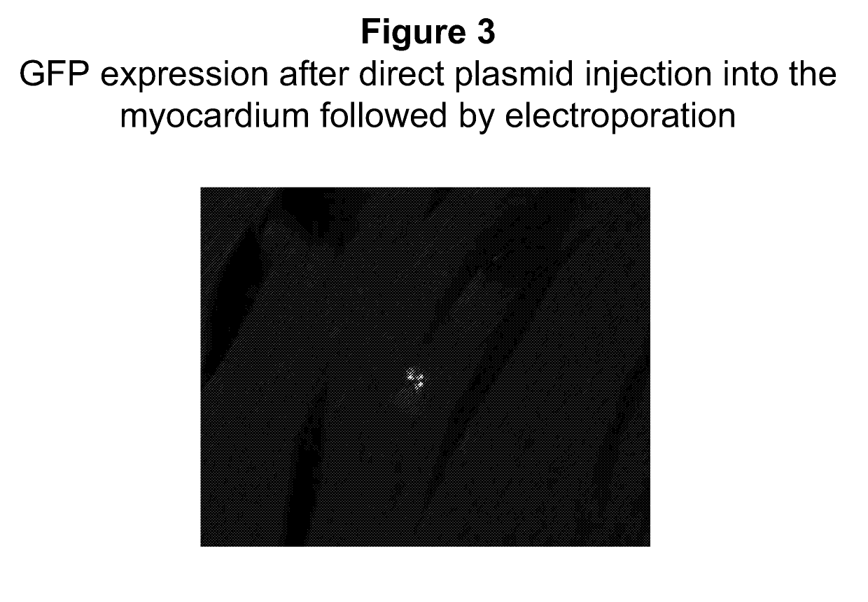

Secreted embryonic alkaline phosphatase assay. Serum samples were thawed and 504, was assayed for SEAP activity using the Phospha-Light Chemiluminescent Reporter Assay Kit (Applied Biosystems, Bedford, Mass.), per manufacturer instructions. The lower limit of detection for the assay is 3 pg/mL. More concentrated serum samples were diluted 1:10 in mouse serum before assaying for SEAP activity. All samples were read using LUMIstar Galaxy luminometer (BMG Labtechnologies, Offenburg, Germany). Animals in group 4, which received the SEAP and GFP under the control of the SP6-39 had the best expression level (FIG. 2 and FIG. 3).

Materials and Equipment:

Defibrillator: Physio-Control LifePak 6s

Sigma Crème—electrode cream (Parker Laboratories, Inc. Fairfield, N.J.—Ref 17-20)

Ultrasound—Aloka 500V (Corometrics Medical Systems, Inc.)

Sonotrack Ultrasound Transmission Gel Medium/Low Viscosity (Echo Ultrasound (800) 233-0261 Reedsville, P.A.)

Terumo Spinal Needle −25G, 3½″ (Burns Vet. Supply—ref. code SN 2590)

Methodology—Pig Trials:

1 mg SEAP (or, 1 mg GFP in a separate experiment) plasmid in 350 μl total volume was injected directly into the heart.

Defibrillate at 100 joules.

All pigs were checked with ultrasound after the defibrillation for cardiac function

### Example 4

### Expression of GFP in Pig Hearts

Heart collection. The pigs from the above-examples were exsanguinated and the apex of the heart was identified. A 2.5 cm square area around the injection site were immediately dissected, and fixed in 1.5% formaldehyde. The dissected area was observed in a darkened room using a UV light at 365 nm wavelength, both at the organ level, and after the tissues were embedded and 5 mm sections cut.

Photographic analysis of the expression area. Samples that demonstrated sufficient fluorescence were photographed using a digital camera, See FIG. 3. Samples with no or very minor fluorescence were not photographed.

1. A method of delivering a biomolecule to a cell that defines an organ, the organ being in situ, comprising:
delivering the biomolecule to the organ, and
providing an amount of energy to the organ effective to electroporate the cells that define the organ.

2. The method of claim 1, wherein the delivering step further comprises:
providing a pharmaceutical formulation comprising the biomolecule for delivery to a subject having the organ.

3. The method of claim 2, wherein the delivering step further comprises:
preparing a delivery apparatus with the pharmaceutical formulation.

4. The method of claim 1, wherein the providing an amount of energy step further comprises:
situating at least one pair of electrodes around the organ,
generating the electroporating amount of energy, and
transmitting the electroporating amount of energy to the pair of electrodes.

5. The method of claim 1, wherein the biomolecule is a polynucleic acid, polypeptide, or pharmaceutical compound.

6. The method of claim 5 wherein the polynucleic acid is a siRNA, linear DNA, or DNA expression construct.

7. The method of claim 5 wherein the polypeptide is a native protein, heterologous polypeptide, or protein fragment.

8. The method of claim 5, wherein the polynucleic acid is a DNA expression construct comprising an encoding sequence.

9. The method of claim 8, wherein the encoding sequence encodes GHRH, IGF-1, caspase, or cytokine.

10. The method of claim 8 wherein the encoding sequence encodes GHRH.

11. The method of claim 8, wherein the DNA expression construct comprises a tissue specific promoter operably linked to the encoding sequence.

12. The method of claim 11 wherein the tissue specific promoter is SPc5-12, SPc1-26, or SPc6-39.

13. The method of claim 1, comprising transdermal, transthoracic, or direct delivery of the biomolecule to the organ.

14. The method of claim 1, further comprising:
imaging the delivery of the biomolecule to the organ.

15. The method of claim 14 wherein the imaging is ultrasound imaging.

16. A method of expressing a heterologous polypeptide in an organ of a subject, comprising:
injecting a needle transdermally, transthoracically, or directly to the organ for delivery of an encoding nucleic acid molecule that encodes the heterologous polypeptide, and
electroporating cells defining the organ by delivering to the cells an amount of energy effective to electroporate the cells.

17. The method of claim 16, further comprising:
providing a pharmaceutical formulation comprising the encoding nucleic acid for delivery to the organ, and
preparing a delivery apparatus with the pharmaceutical formulation, said delivery apparatus being in fluid communication with said needle.

18. The method of claim 16, wherein the electroporating step further comprises:
situating at least one pair of electrodes around the organ,
generating the electroporating amount of energy, and
transmitting the electroporating amount of energy to the electrodes.

19. The method of claim 16, wherein the encoding nucleic acid encodes GHRH, IGF-1, caspase, or cytokine.

20. The method of claim 16, wherein the encoding nucleic acid
comprises a tissue specific promoter operably linked to the encoding sequence.

21. The method of claim 20, wherein the tissue specific promoter is SPc5-12, SPc1-26, or SPc6-39.

22. The method of claim 16, further comprising:
imaging the delivery of the biomolecule to the organ.

23. The method of claim 22 wherein the imaging is ultrasound imaging.

24. A system adapted for the delivery of an encoding nucleic acid molecule to an organ of a mammal for the expression of a polypeptide from cells defining the organ, comprising:
a needle for delivery of the encoding nucleic acid molecule to the organ, and
an electorporation device capable of delivering pulses of energy at an intensity effective for electroporation of the cells of the organ.

25-28. (canceled)
